(19) United States
(12) Patent Application Publication
(10) Pub. No.: US 2026/0341449 A1
(43) Pub. Date: Oct. 29, 2026

(54) AI-Driven Prescription Management System

(71) Applicant: HR Pharmaceuticals, Inc., York, PA (US)

(72) Inventor: Mathew Hanna, Lancaster, PA (US)

(73) Assignee: HR Pharmaceuticals, Inc., York, PA (US)

(21) Appl. No.: 19/237,707

(22) Filed: Jun. 13, 2025

Related U.S. Application Data
(60) Provisional application No. 63/796,825, filed on Apr. 29, 2025.

(51) Int. Cl. G16H 20/10; G06Q 30/018; G16H 10/60; G16H 15/00; H04L 9/40

(52) U.S. Cl. CPC G16H 20/10; G06Q 30/018; G16H 10/60; G16H 15/00; H04L 63/0428; H04L 63/166

(57) ABSTRACT

A prescription/order management system includes a processor and a server configured with a GPU to execute AI algorithms and optical character recognition, including natural language processing and machine learning models a computing device configured to run a web-based application accessible via a network a panel having a setup new clinic module enabling participant access by allowing administrators to onboard healthcare facilities with details like clinic name and address, the panel further having oversight privileges over a back-end user and a front-end user to monitor system performance and manage user accounts. The system includes a back-end user module accessible by the back-end user having an orders module for receiving, tracking and fulfilling prescription/orders, including categorizing orders by status and displaying order details. The system includes a front-end user module accessible by the front-end user having a new form module that enables the front-end user to create a new prescription/order form with assisted data entry that leverages generative artificial intelligence and optical character recognition, where a machine learning model extracts data from uploaded documents to pre-fill form fields. The system includes a validation module, the validation module compiling product information and qualifications by leveraging an inputted submission of the front-end user through the new form module to prepare a work order to present to the back-end user during an ordering process of the specific product and generating a medical record addendum with structured data, including HCPC codes, product selection, patient information, diagnosis, usage frequency and qualification, to ensure compliance.

### CROSS-REFERENCE TO RELATED APPLICATIONS

The application claims priority to U. S Application No. 63/796,825, filed on Apr. 29, 2025.

### FIELD OF THE INVENTION

The present invention pertains to systems and methods for automating prescription order completion, validation, processing and fulfillment. Specifically, it leverages generative artificial intelligence (AI), such as natural language processing (NLP) to parse unstructured medical documents, machine learning to predict and correct incomplete data entries, and rule-based algorithms to ensure compliance with regulatory standards and documentation requirements like promulgated and/or set forth by Centers for Medicare and Medicaid Services (CMS), as amended from time to time, with respect to prescriptions/orders for intermittent catheters, indwelling (Foley) catheters, external catheters, urological accessories, and related products/supplies. The system streamlines the prescription/order process by automating data capture and validation, reducing errors, and generating structured medical documentation, such as medical record addendums, to facilitate seamless communication between healthcare providers, vendors, and patients. The system is particularly suited for managing durable medical equipment (DME) prescriptions/ orders, such as surgical supplies, and supports interactions among prescribers, medical suppliers, manufacturers insurance payers, patients and other third parties. The system may be used to cover durable medical equipment, prosthetics, orthotics and medical supplies (DMEPOS, CMS definition). Additionally, the system may be used to cover ostomy, urological, wound care, diabetes, enteral feeding, etc.

### BACKGROUND

Traditional digital urological supplies prescription/order management workflows and systems do not solve the requirement of manual data entry, face challenges with respect to ensuring the prescription/order is compliant with CMS documentation requirements to support medical necessity and avoid waste/abuse and fail to deliver the promised efficiency over paper documentation due to various stakeholders involved correcting incomplete or inaccurate information. These inefficiencies lead to healthcare professionals spending greater time on prescription/order processing and often having to correct documentation associated with the prescription/order to maintain compliance with CMS requirements and support medical necessity, which, in turn, delays, adversely impacts patient care and delays fulfillment of the prescription/ order. Existing solutions, including electronic health record (EHR) systems, DME supplier management software systems, DMEPOS order management software and websites, and DME supplier websites with form-based urological supply order functions lack the capability to dynamically capture pertinent patient demographic information from medical documentation for generating a urological supply prescription/order or validate an urological supply prescription/order for compliance with CMS requirements to support medical necessity and avoid waste/abuse to reduce or eliminate inefficiencies associated with correcting documentation associated with a urological supply prescription/order Prior art systems often rely on static, form-based templates or manual workflows, lacking generative artificial intelligence and optical character recognition to capture pertinent patient demographic information to streamline the data entry process with respect to preparing a urological supply prescription/order. These systems also fail to validate the medical records associated with the urological supply prescription/order to ensure the medical record is amended to include the proper verbiage to support the medical necessity for the prescribed urological product, leading to repeated back-and-forth between healthcare professionals and industry representatives and delay in processing. Therefore, there is a need for a solution that provides: 1) based on the information input by a healthcare professional, validating the medical documentation to ensure it contains the proper verbiage, as required by CMS, to support medical necessity and prevent waste/abuse, and if needed, generate a real-time amendment to the medical record 2) streamlining data entry to drive efficiency in workflow and reduction in human error and 3) enhancing the speed of urological supply prescription/order processing using generative artificial intelligence and optical character recognition. The present invention offers a novel platform integrating generative artificial intelligence optical character recognition, and validation processes to address the inefficiencies of urological supply prescription order management. The present invention generally provides automated data validation using artificial intelligence algorithms to detect and correct errors in real time, achieving a reduction in error rates by up to 50% compared to manual processes, as demonstrated by internal testing with a dataset of 5,000 prescription orders. The system may use intelligent document processing by extracting text, handwriting, tables, and layout elements from scanned documents using machine learning. The system may support forms, invoices, IDs, and more, providing structured data like key-value pairs or tabular data. The extracted data is used for tasks like document classification, summarization, Q&A, or generating structured outputs.

The present invention further provides seamless integration by interoperable modules allowing direct communication between doctors, providers, and patients through a secure, web-based interface that ensures data privacy and compliance with HIPAA. The present invention further utilizes the prescribing information input by the healthcare provider and deploys a validation process to ensure all required information is contained in the prescription/order and the medical records contain CMS required verbiage to support medical necessity and prevent waste/overuse, generating an automated, selection based medical record addendums to ensure the medical record(s) meet the requirements for the urological supply order and streamline vendor communication. The automated, selection-based addendum compiles patient information, date of office visit, HCPCS code information, product qualifications, diagnosis codes, and usage frequencies into a structured document, reducing the need for iterative communication between prescribers and vendors and accelerating fulfillment timelines for urological supply orders.

### SUMMARY

A multi-faceted generative artificial intelligence and optical character recognition driven urological supply prescription/order management system includes a processor and a server configured with a graphics processing unit (GPU) to execute AI algorithms and optical character recognition, a computing device configured to run a web-based application accessible via browsers like Chrome, Safari, or Firefox, and a database server hosting a relational database, such as PostgreSQL, to store prescription/order, patient, and compliance data. The system further includes an administrative/navigation panel having a setup new clinic module enabling participant access, which allows users to onboard new healthcare facilities by entering details such as clinic name, address, and contact information, the administrative/navigation panel further having oversight privileges over a back-end user and a front-end user to monitor system performance, manage user accounts and designated prescribers with NPI numbers, and ensure compliance with operational protocols. The system further includes a back-end user module accessible by the back-end user having an order module for tracking and fulfilling prescriptions/orders, including categorizing orders by status (e.g., submitted, pending, fulfilled) and providing visibility into order details like patient name and clinic. The system further includes a front-end user module accessible by the front-end user having a new form module that enables the front-end user to create a new order prescription/order with generative artificial intelligence and optical character recognition assisted data entry, where the system extracts information from uploaded documents, such as face sheets or chart notes, to pre-fill form fields like patient demographics, insurance information, and/or diagnosis codes. The system enables a user submitting a prescription/order for urological supplies to select the appropriate product for the patient using either the Quick Select feature, which only requires the user to confirm a few desired product attributes for patient, or Market Place, which incorporates a unique shopping-cart-like experience to the ordering process to guide the user through product selection for the patient. The system further includes a validation module, the validation module compiling product and prescribing information by leveraging an inputted submission of the front-end user through the new prescription/order module to prepare a work order to present to the back-end user during an ordering process of the specific product and generating a medical record addendum with structured data, including patient information, date of office visit HCPC code(s) usage frequency, justification to support medical necessity, and other pertinent information to ensure compliance with CMS requirements for urological supplies, and which is then presented to the prescriber for review, confirmation, and signature. The generative artificial intelligence and optical character recognition integrates across various modules, using a shared machine learning model to extract data, validate inputs, and generate compliance documents, ensuring consistency and reducing processing time. The system employs a cloud-based architecture with load balancing to support scalability across multiple clinics with no threshold of the number of clinics, handling thousands of transactions daily with minimal latency. The system utilizes AI/OCR are used to extract; validation is a process and leverages AI/OCR extracted data but does not use AI/OCR for the specific process.

A prescription/order management system includes a processor and a server configured with a GPU to execute AI algorithms and optical character recognition, including natural language processing and machine learning models a computing device configured to run a web-based application accessible via a network a panel having a setup new clinic module enabling participant access by allowing administrators to onboard healthcare facilities with details like clinic name and address, the panel further having oversight privileges over a back-end user and a front-end user to monitor system performance and manage user accounts. The system includes a back-end user module accessible by the back-end user having an orders module for receiving, tracking and fulfilling prescription/orders, including categorizing orders by status and displaying order details. The system includes a front-end user module accessible by the front-end user having a new form module that enables the front-end user to create a new prescription/order form with assisted data entry that leverages generative artificial intelligence and optical character recognition, where a machine learning model extracts data from uploaded documents to pre-fill form fields. The system includes a validation module, the validation module compiling product information and qualifications by leveraging an inputted submission of the front-end user through the new form module to prepare a work order to present to the back-end user during an ordering process of the specific product and generating a medical record addendum with structured data, including HCPC codes, product selection, patient information, diagnosis, usage frequency and qualification, to ensure compliance.

### BRIEF DESCRIPTION OF DRAWINGS

In the following, the present invention is described in more detail with references to the drawings in which:

FIG. 1 illustrates an exemplary schematic of the hardware architecture, including a web server, database server, network infrastructure, and client computing devices.

FIG. 2-6 illustrate an exemplary administrative panel of the invention, including interfaces for setting up new clinics, adding users, and monitoring pending clinics.

FIG. 7-10 illustrate a plurality of exemplary back-end user modules, including dashboards for system metrics, order tracking interfaces, and clinic management tools.

FIG. 11-32 illustrate a plurality of exemplary front-end user modules, including interfaces for creating prescriptions/order, selecting medical products, generating written orders, and validating order inputs for compliance and producing medical record addendums as needed.

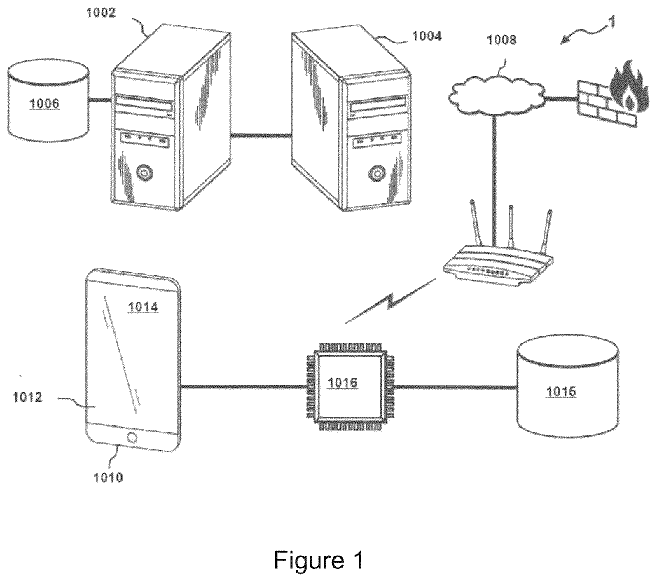

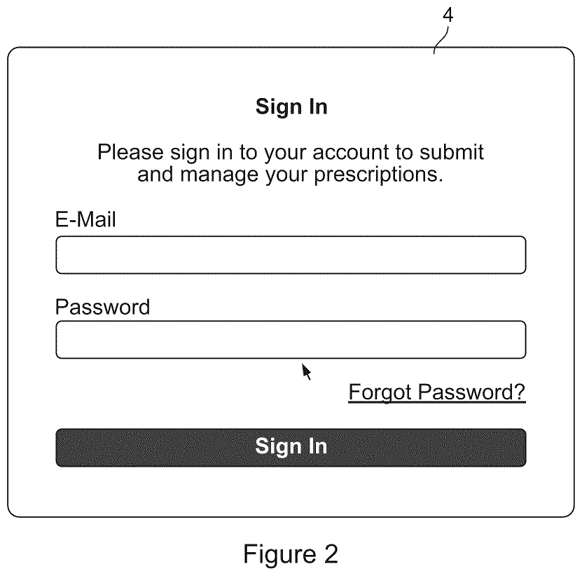

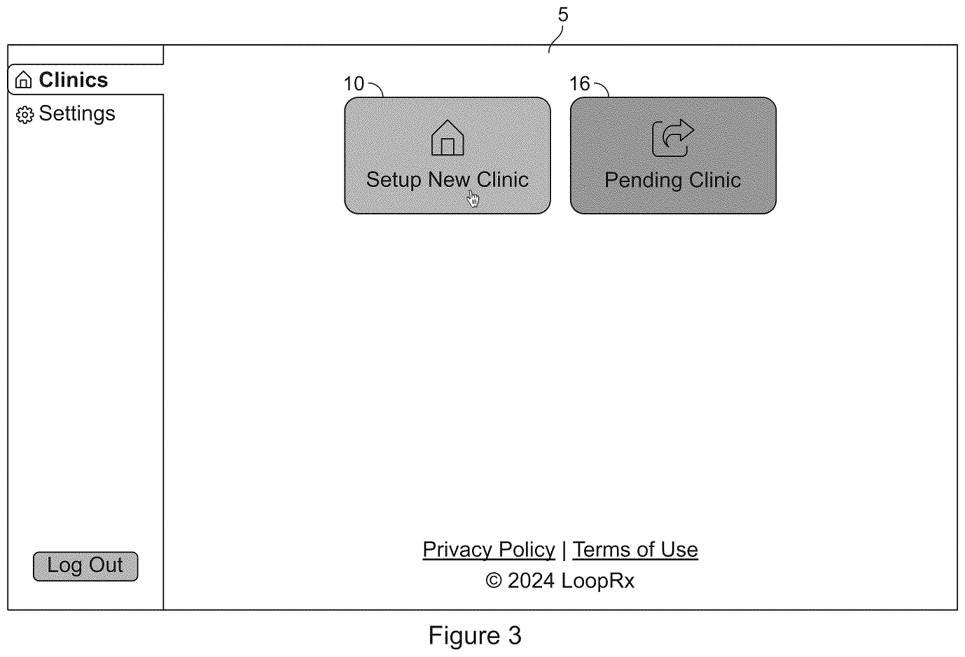

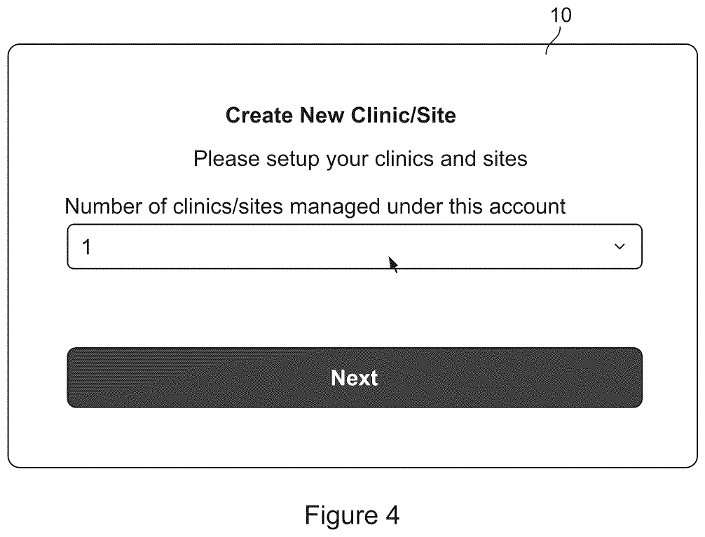

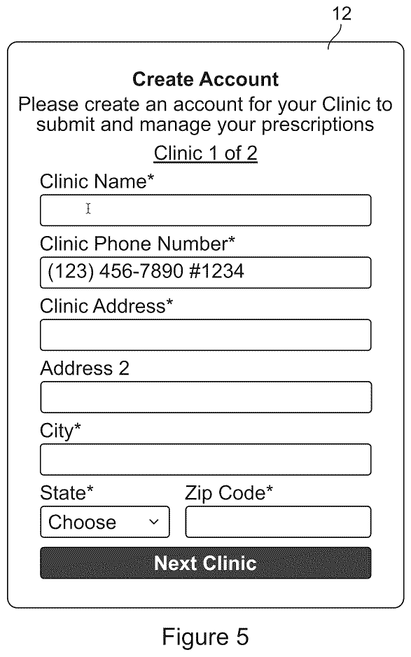

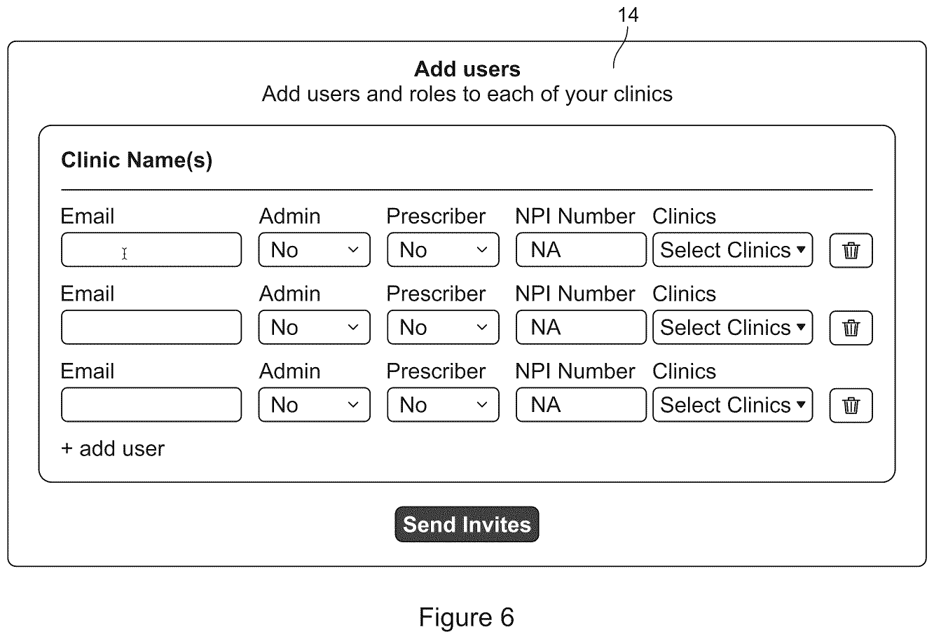

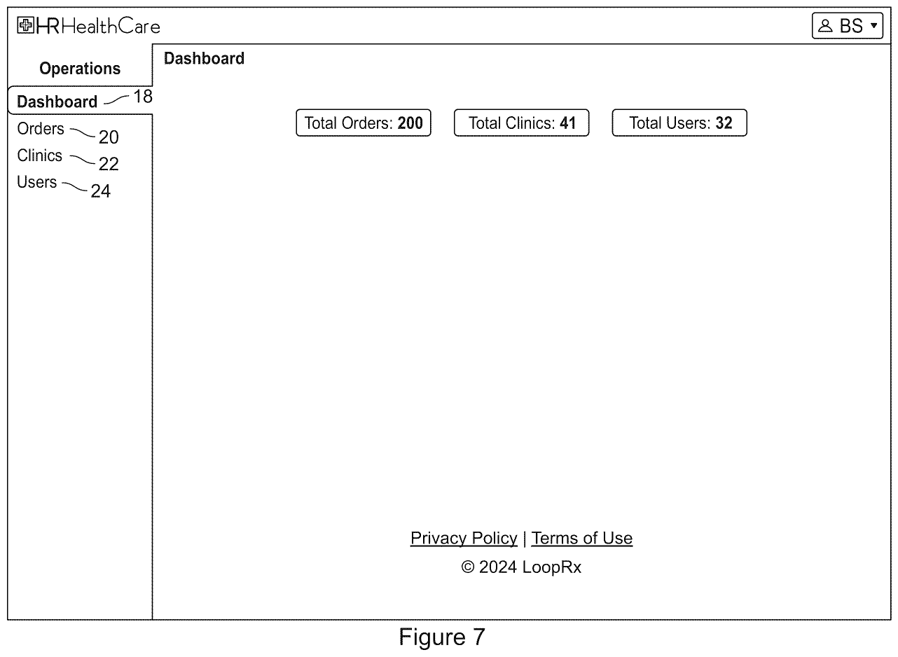

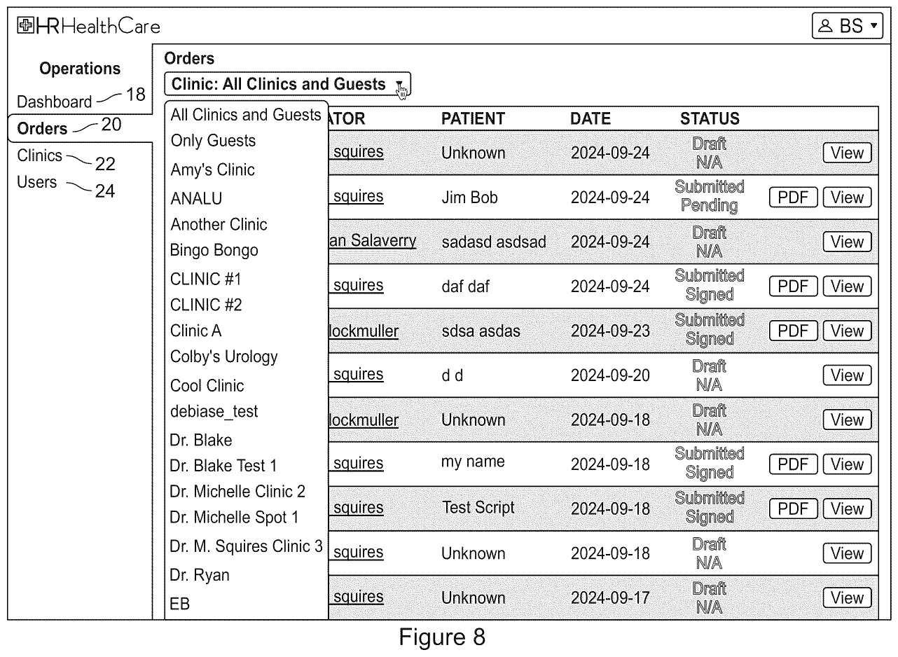

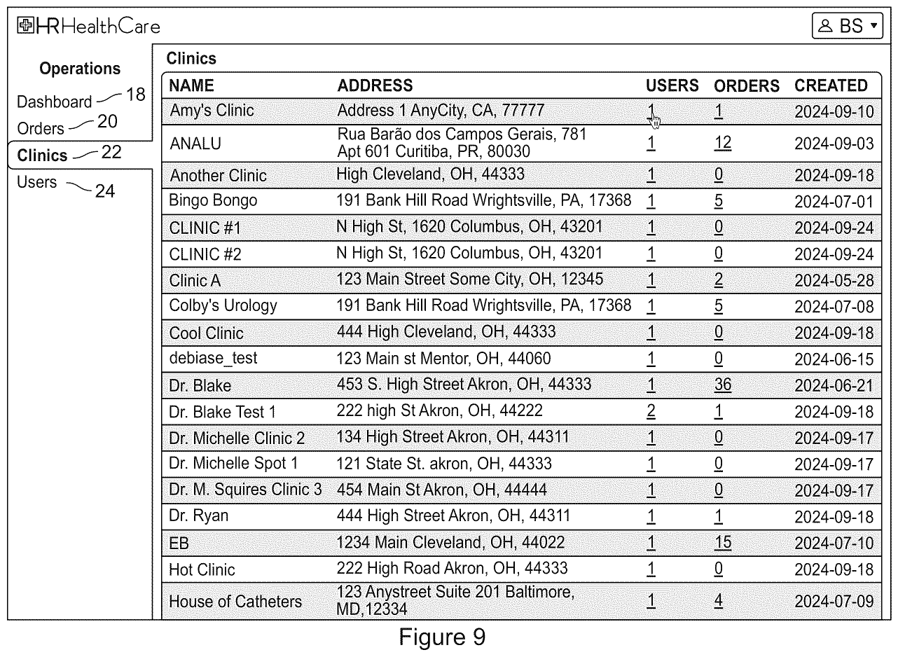

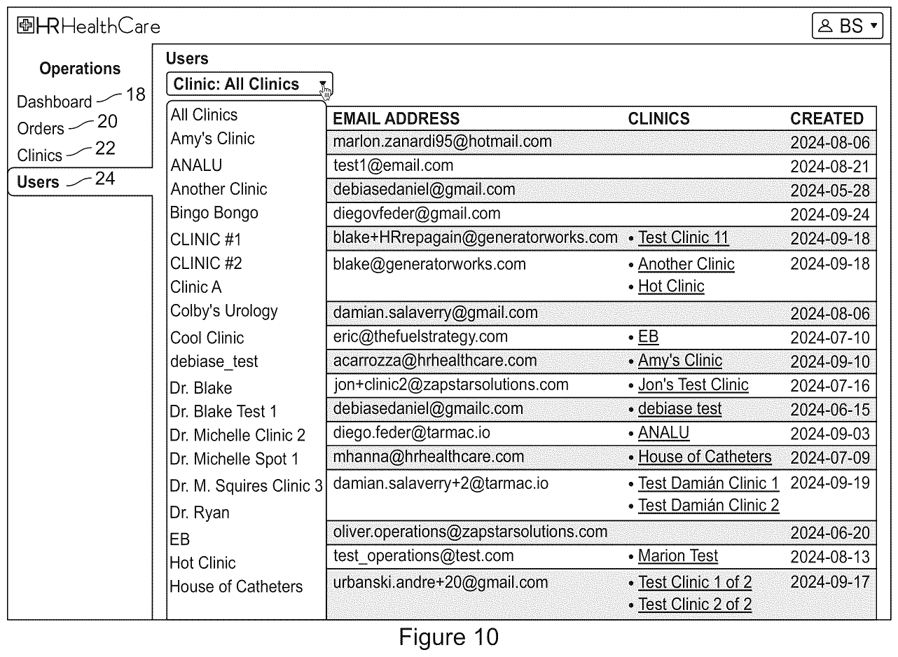

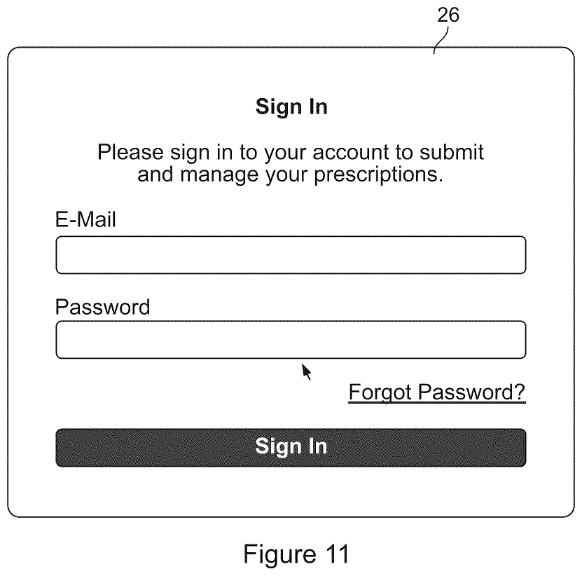

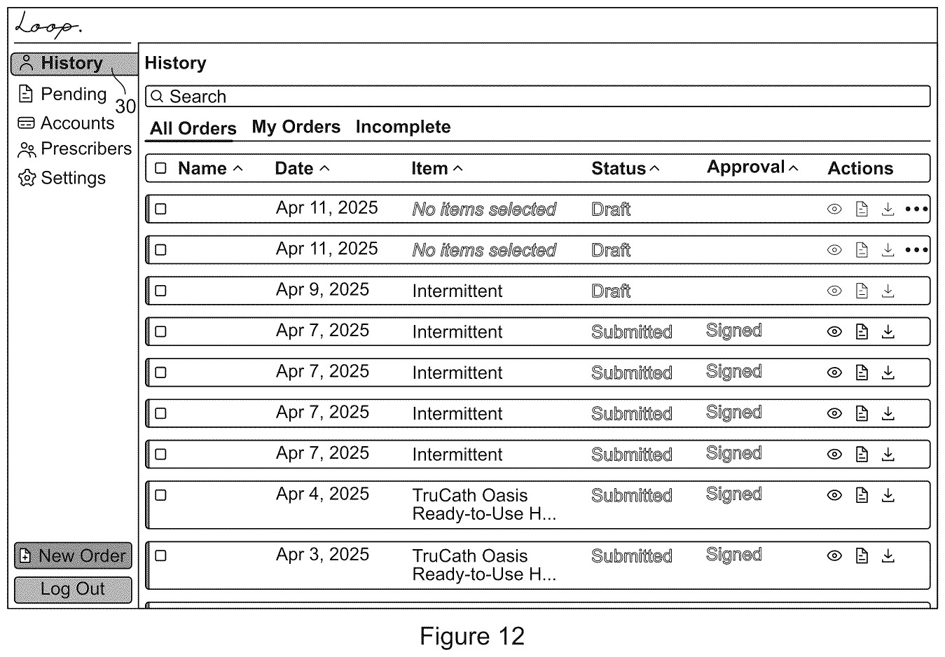

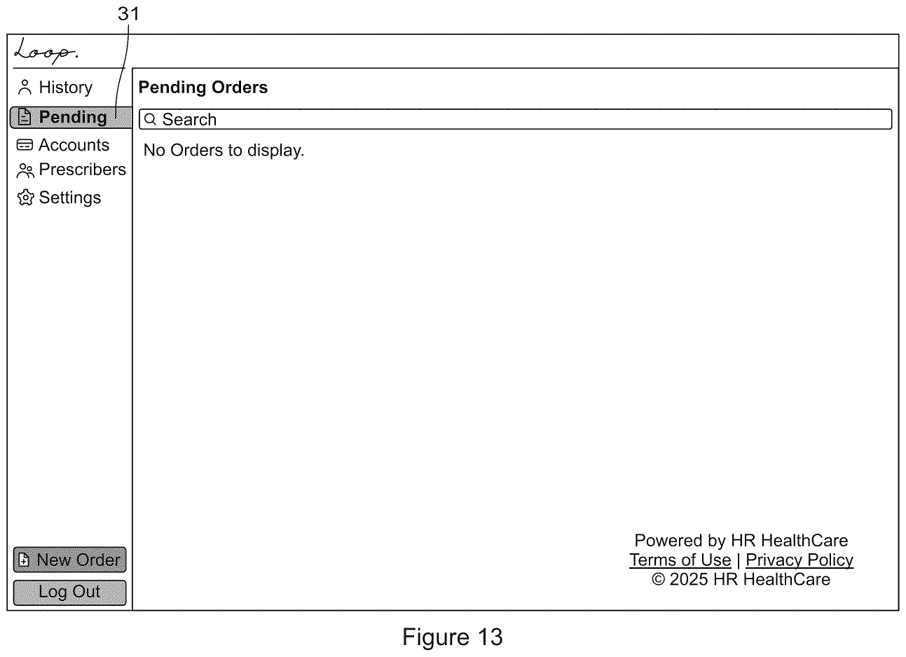

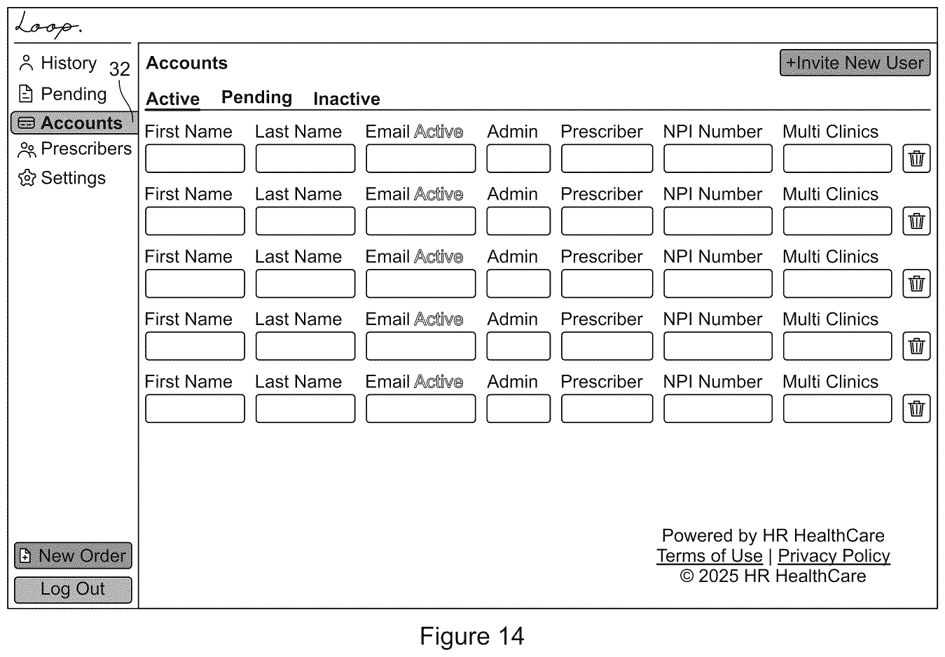

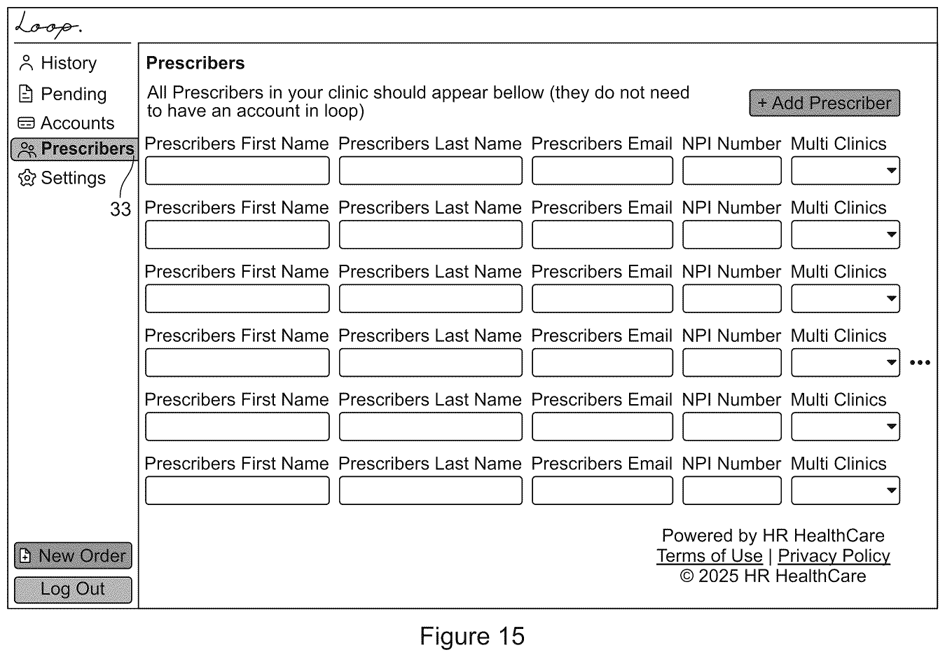

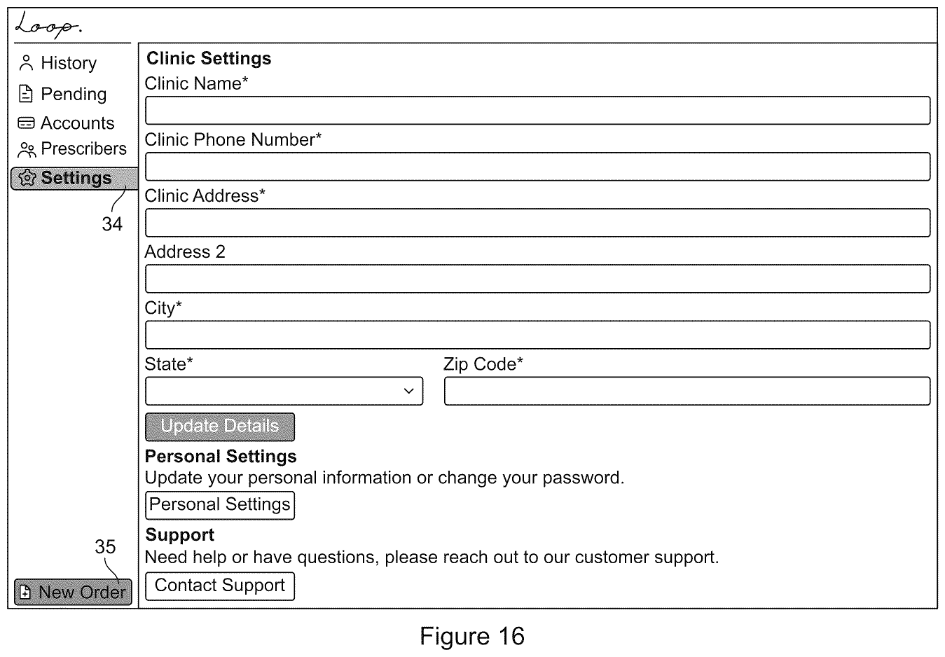

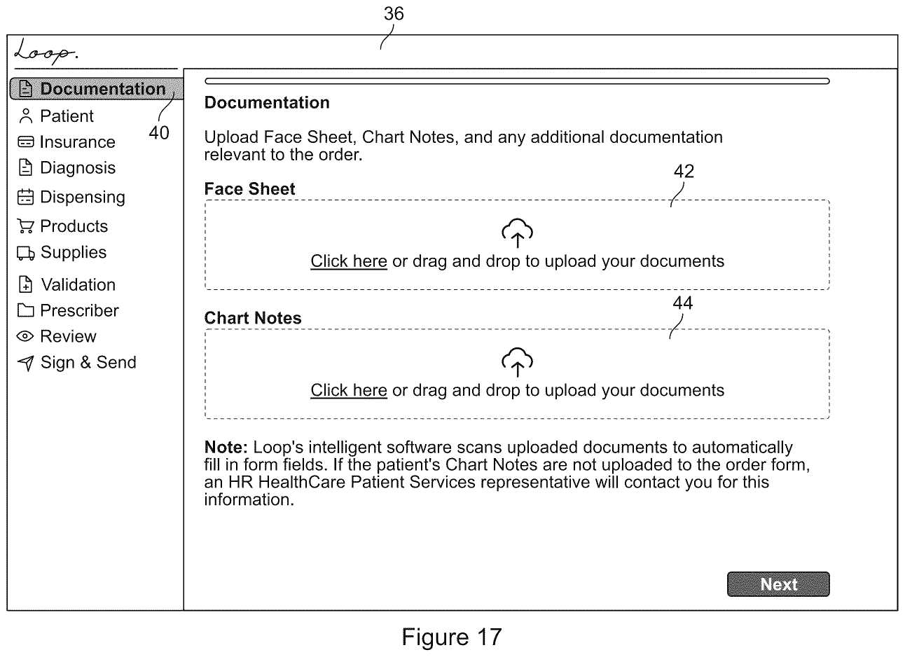

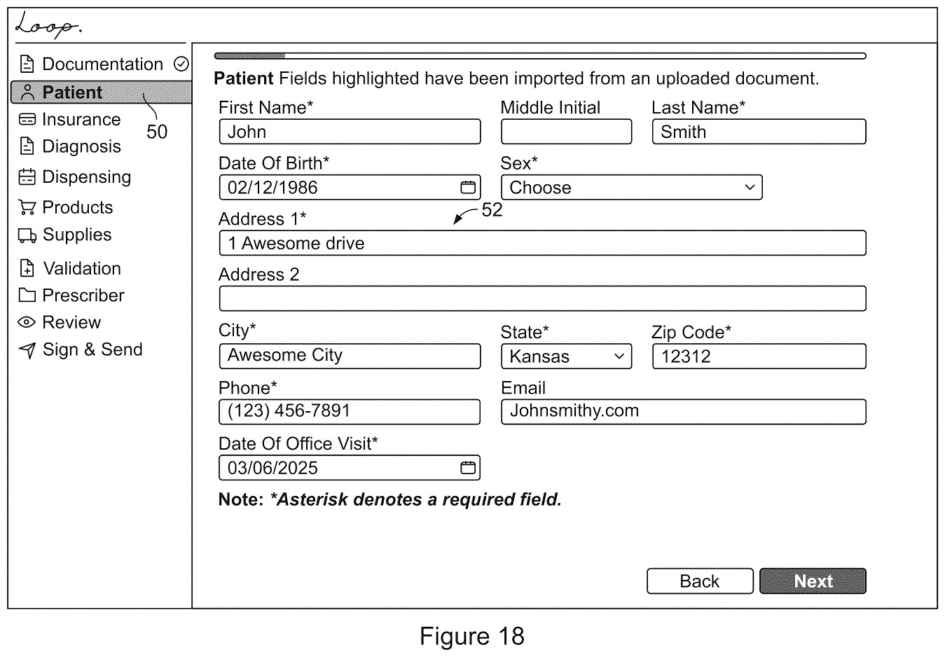

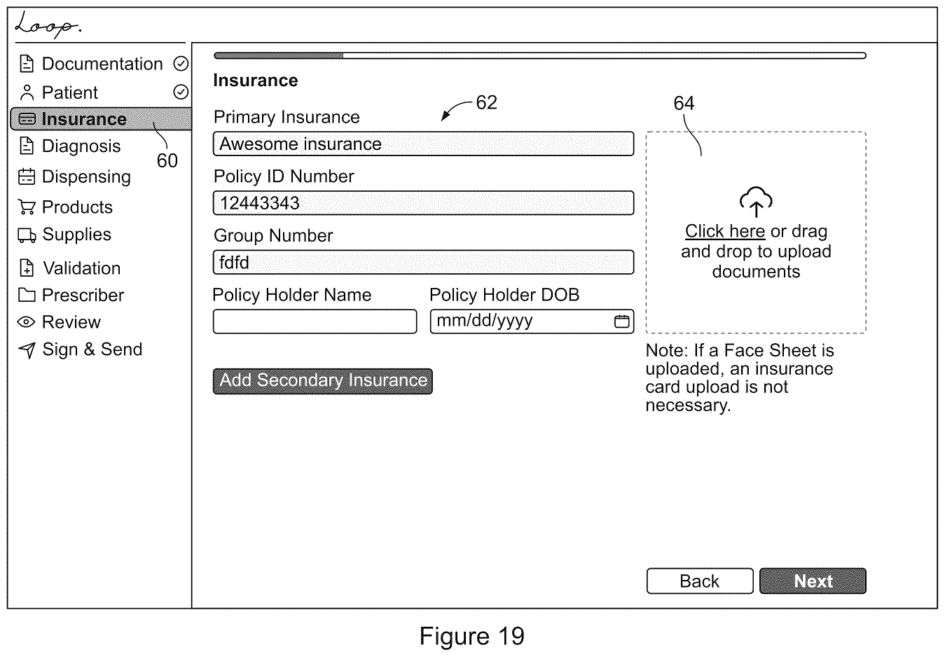

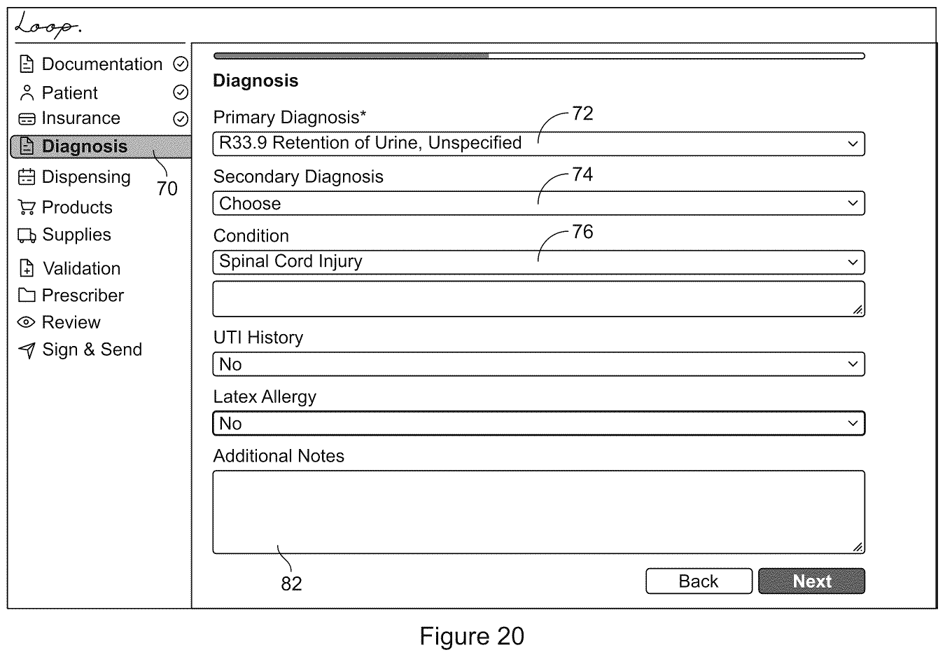

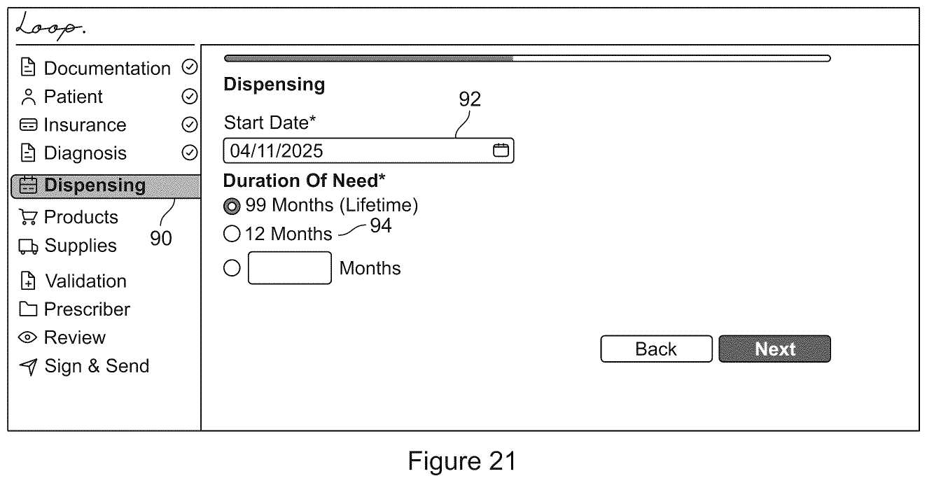

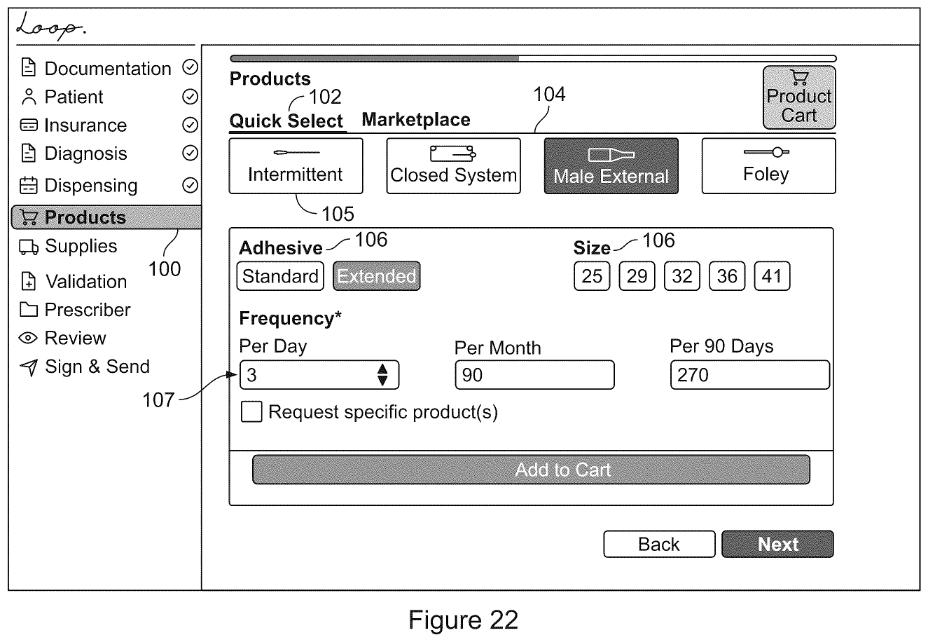

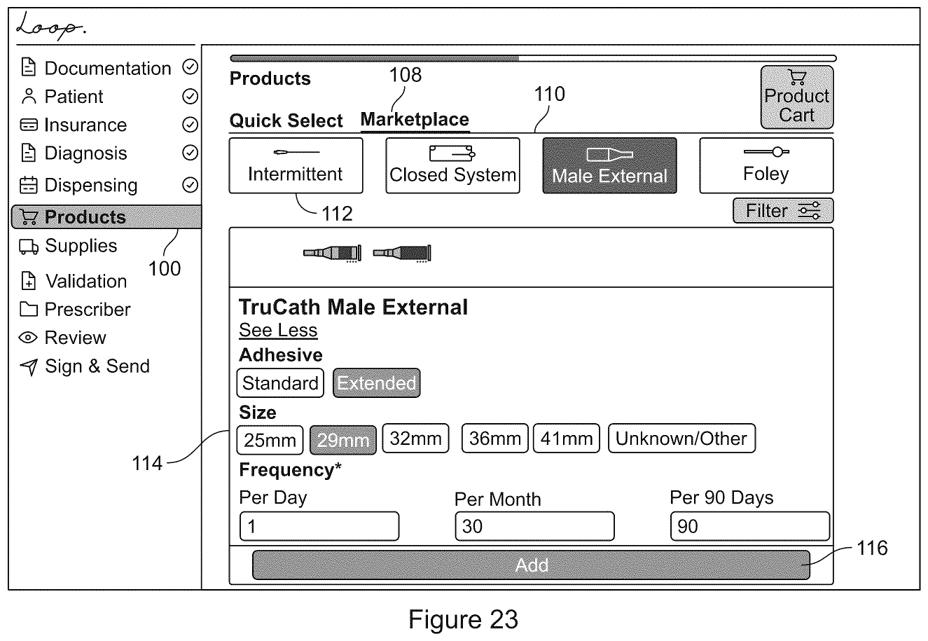

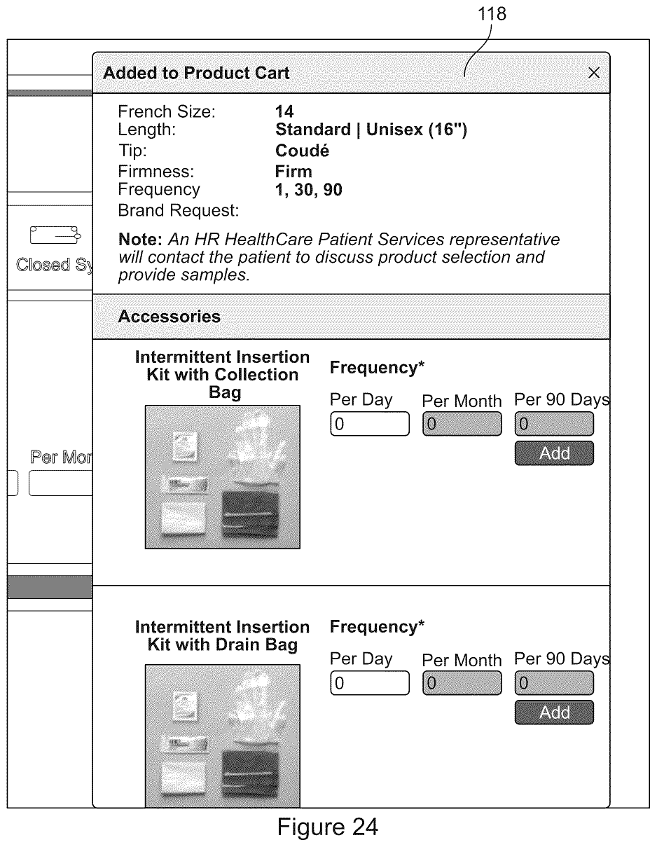

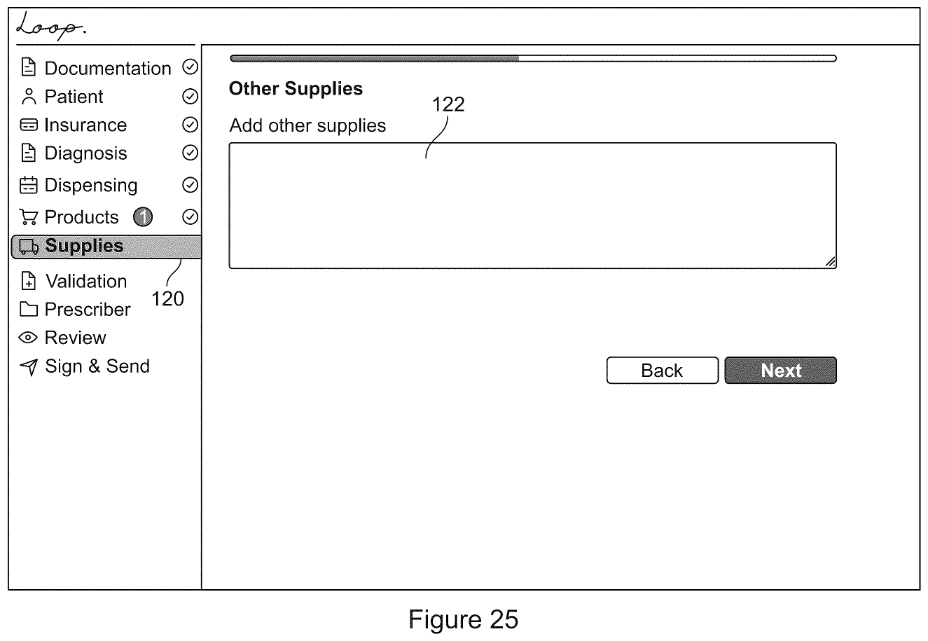

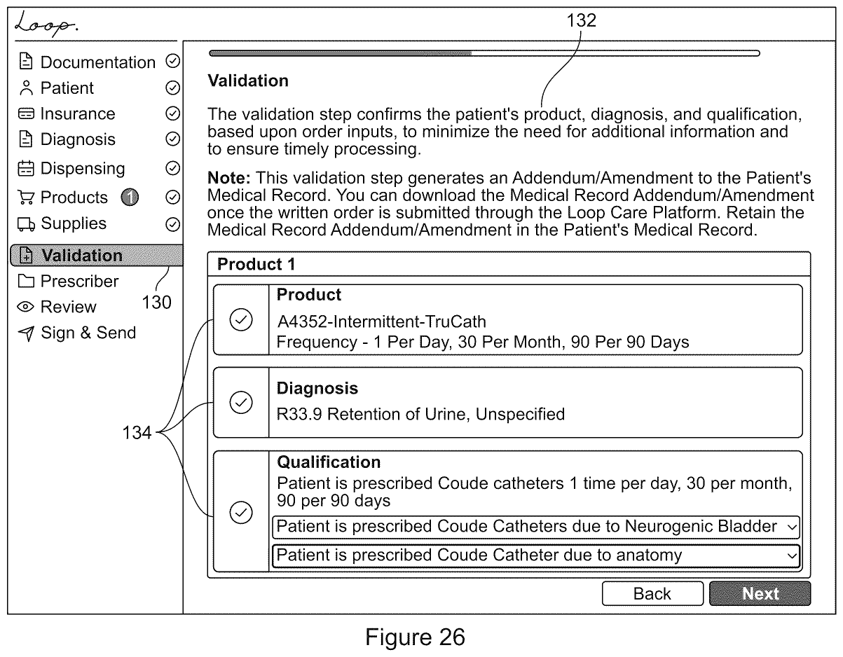

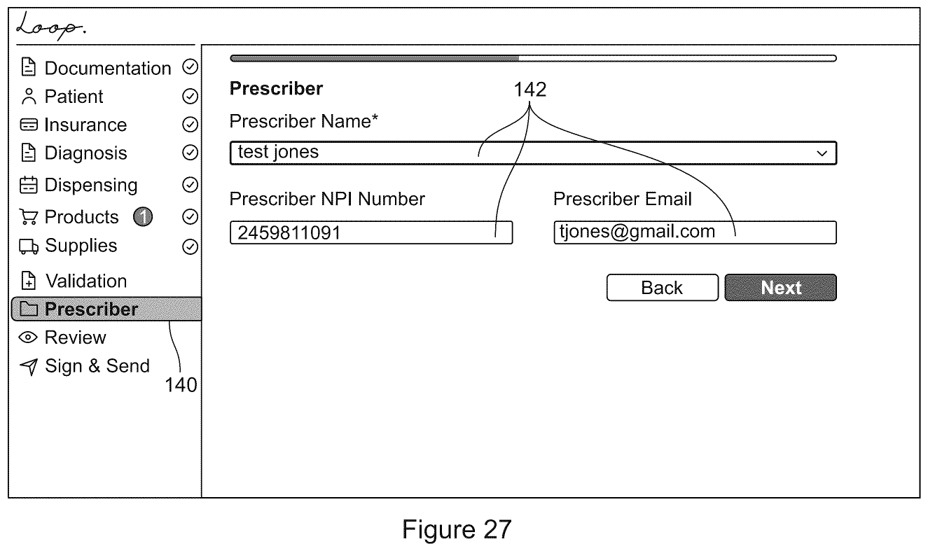

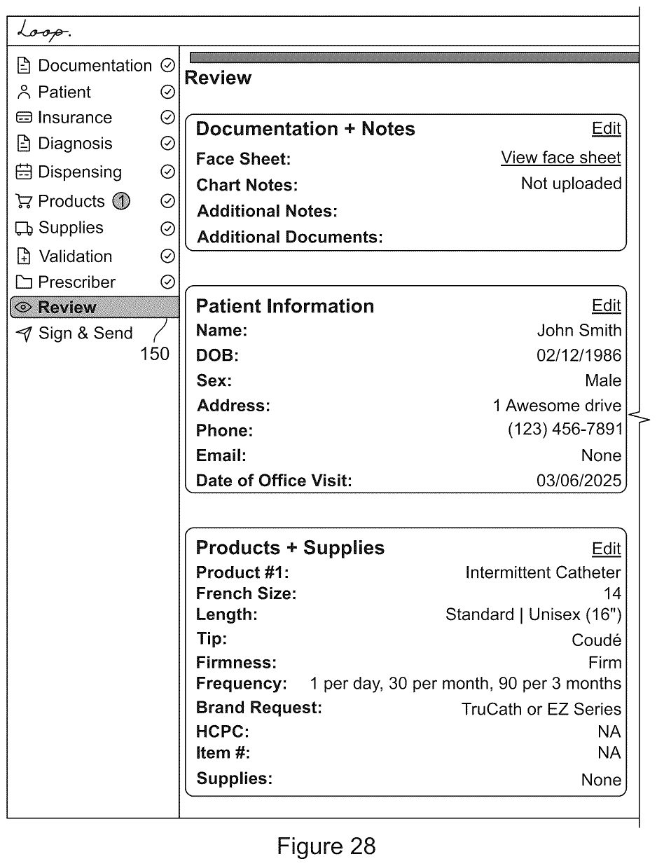

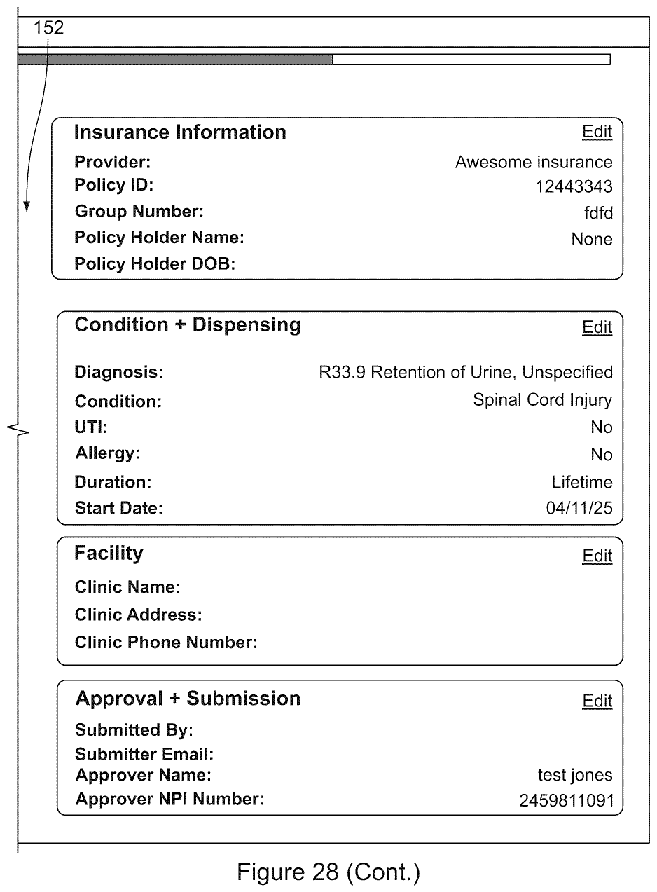

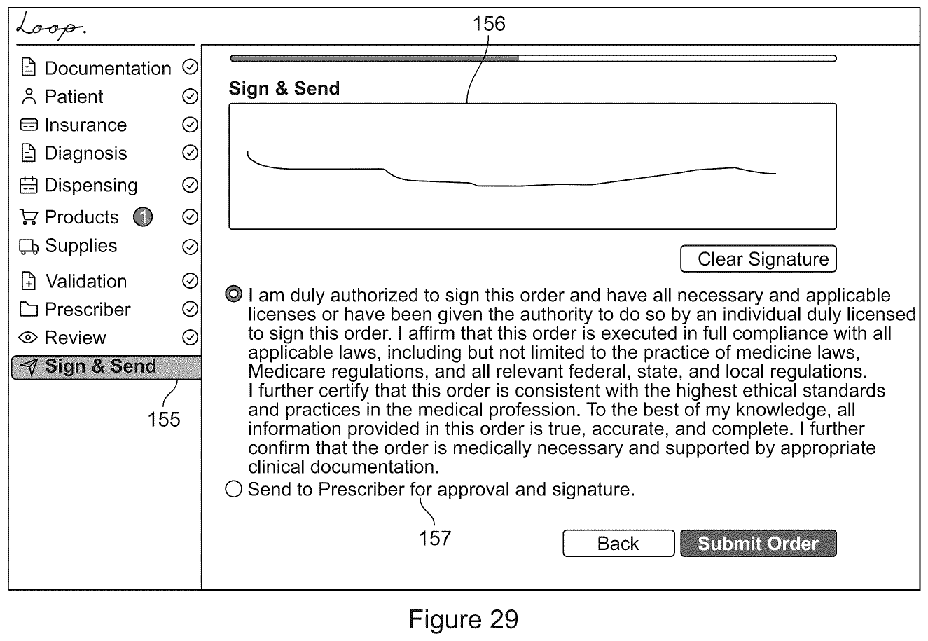

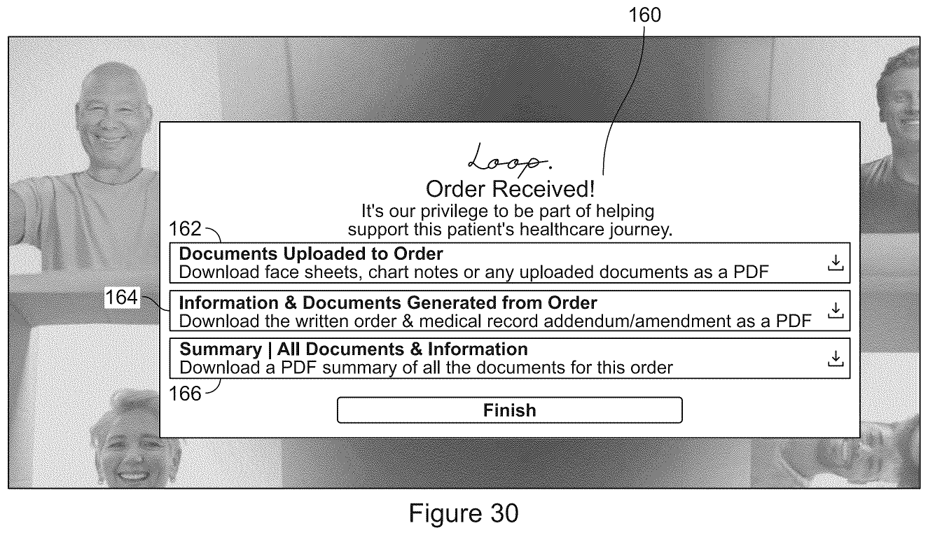

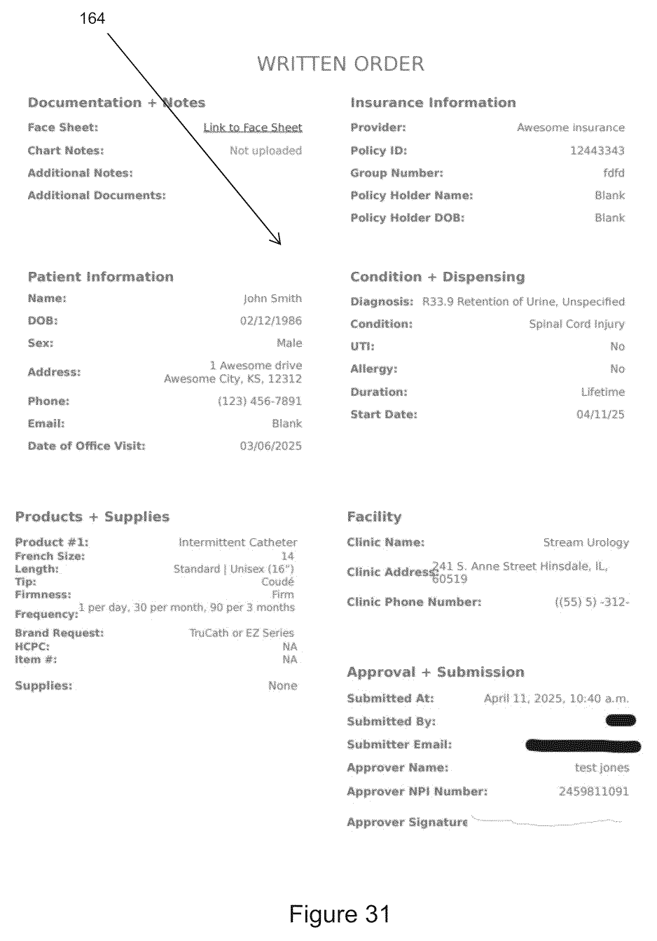

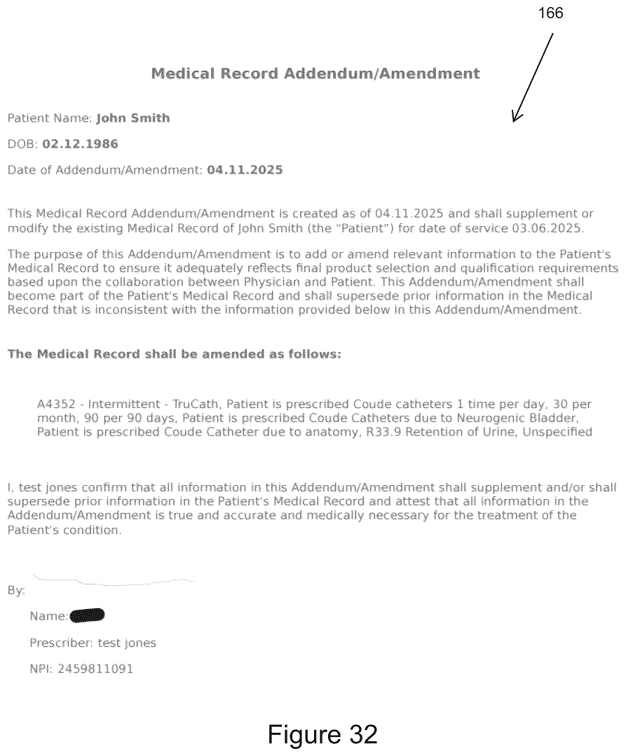

### DETAILED DESCRIPTION OF THE EMBODIMENTS

In an exemplary embodiment, the teachings herein describe a prescription/order management system 1 that allows for a streamlined experience for all stakeholders, from healthcare professionals to catheter suppliers to patients. The system is a valuable asset in medical prescription/order management by automating the prescription/order process, reducing manual errors, ensuring compliance with regulatory requirements and accelerating fulfillment timelines.

In the exemplary embodiment, the design of the system 1 is centered around providing a robust, efficient, and user-friendly software platform for managing urological supply prescriptions/orders, reducing inefficient back-and-forth between exchanges between prescribers and suppliers by leveraging data inputs to ensure the prescription/order is compliant with regulatory requirements/guidelines prior to submission through the platform, which in turn promotes faster processing of the prescription/order for the patient. The administrative/navigation panel 5 provides a centralized interface for system oversight, allowing administrators to configure clinics, manage user permissions, and monitor system performance metrics. The back-end user module enables efficient operational management of prescription/orders, by offering tools to track order status, view clinic-specific data, and manage front-end user accounts. The back-end user may be a supplier/manufacturer who fulfills the order, receiving automated prescription/orders that include validated product details and compliance documentation.

The front-end user module 8 ensures that front-end users, typically healthcare providers or clinic staff, have access to an intuitive and automated interface for creating, managing, and submitting urology supply prescriptions/orders. Together, these elements create a seamless workflow that leverages generative artificial intelligence optical character recognition and validation processes to reduce manual input and improve the overall efficiency of the prescription process. The system 1 is an innovative solution addressing critical inefficiencies in urology supply prescription/order processing. By integrating generative artificial intelligence and optical character recognition with robust process-driven system architecture, it ensures accurate, efficient, and compliant urology supply prescription/order submission processing and, fulfillment, benefiting healthcare providers, suppliers, and patients alike. For example, the system utilizes data input by the healthcare professional to validate the urology supply prescription/order for compliance with CMS regulations and generates a Medical Record Addendum/Amendment that is reviewed and signed by the healthcare professional, a structured document that compiles patient information, office visit date, product qualifications to support medical necessity, diagnosis codes, HCPCS codes, and usage frequency. The validation process and corresponding Medical Record Addendum/Amendment ensures compliance with regulations promulgated by CMS, as amended from time to time, with respect to urological supply prescriptions/orders and virtually eliminates the need for back-and-forth communication between healthcare professionals and industry representatives to correct or update documentation to support medical necessary for the prescribed product. One skilled in the art would understand that deployment in other healthcare applications, such as lab test ordering, infusion ordering or other DMEPOS ordering, could be utilized.

The system comprises a web-based application hosted on a secure server infrastructure, accessible via a network by client computing devices, such as desktops, tablets, or smartphones. In the exemplary embodiment, the application site is designed to work on Internet Explorer 10+, and the most up-to-date versions of Chrome, Safari, and Firefox across all major operating systems and updated device platforms, for example, Android and iOS. The system supports responsive design to ensure usability across devices, with a minimum screen resolution of 1024×768 pixels for optimal performance.

Hardware infrastructure for an embodiment of the system will be described. In an exemplary embodiment, the system is built on a network router (for instance, a wireless router with 802.11ac or later standards) and connected to a database server, while also utilizing known hardware components, including a web server, a firewall for securing data transmissions, a network, and the computing device 1010. The system requires a web server with at least 16 GB of RAM, a quad-core processor, and a GPU (e.g., NVIDIA A100) to handle AI computations, such as natural language processing and machine learning inference. The database server, hosting a relational database like PostgreSQL, that is HIPAA compliant requires at least 1 TB of storage to accommodate prescription/order records, patient data, and compliance logs, with scalability to support hundreds of clinics and thousands of daily transactions.

The hardware infrastructure includes a plurality of integrated system, HIPAA-compliant servers, including a web server to host the application and a database server to manage data storage and retrieval, with one or more databases 1006 (e.g., a PostgreSQL database with tables for patients, orders, and compliance mappings), a network interface 1008 accessible through various known communication protocols, such as TCP/IP, cellular protocols including GSM, Wi-Fi, Wi-Max, or other wireless communications technologies or combination of wired or wireless channels, network security devices, such as firewalls to prevent unauthorized access and ensure HIPAA compliance, and a computing device 1010 having a processing unit 1016 (e.g., a CPU or GPU with at least 2.5 GHz clock speed) and memory 1015 (e.g., at least 8 GB RAM for client devices). The system 1 uses a secure application programming interface (API) based on RESTful architecture to facilitate communication between the web server and database server, ensuring data integrity and privacy. For example, all data transmissions are encrypted using TLS 1.3, and user authentication employs OAuth 2.0 protocols.

The system 1 incorporates fault-tolerant mechanisms, including redundant servers and automated backups to a secondary database, ensuring 99.9% uptime for critical healthcare operations. The AI algorithms are deployed using a containerized framework, such as Docker, with Kubernetes for orchestration, enabling efficient scaling and updates.

The system allows 1 a user to access a plurality of system files that include data, such as information and images, through the computing device 1010 and network traffic information on the database server (e.g., SQL Server or PostgreSQL) that connects to a web server. The web server functions as a way for the network interface to communicate to the database server through a known application-programming interface (API) between the computing device 1010 and the database server 1002. A firewall may be used for security purposes, such as blocking unauthorized access to the web server 1004 and permitting unauthorized communication thereto. The system 1 is designed to run the computing device 1010 through modules that can be downloaded over personal area networks (PANs), local area networks (LANs), campus area networks (CANs), wide area networks (WANs), metropolitan area networks (MANs), and any new networking system developed in the future. One skilled in the art should appreciate that the system can be maintained solely through the computing device, as the system modules can be pre-loaded to the computing device. The computing device has a processor for performing the necessary calculations and performing the actions for the system module having a plurality of service modules within the module, and also having memory, configured for storing and accessing the system files.

In the shown embodiment, the user may connect to the network interface using the computing device through the router, for instance. One skilled in the art would appreciate that other hardware and protocol designs are possible as long as such modifications would not divert from the spirit of the invention.

In an exemplary embodiment, the computing device generally includes the general user interface, the memory device, and the processor. In the shown embodiment, the computing device is a tablet computer or mobile phone with a touchscreen display 1012. The touchscreen display uses finger or stylus gestures to navigate the general user interface (GUI) 1014. However, one skilled in the art should appreciate that other implements could be used, including a computer mouse, a keyboard, or joystick. In fact, one skilled in the art should appreciate that the computing device is a physical computer and could be, but not limited to, a desktop computer, a laptop computer, or a cell phone/tablet, and utilize a downloaded app or web browser. The memory device is a storage device having computer components and recording media used to retain digital data. The processor is a central processing unit (CPU) that manipulates data stored in the memory device by performing computations.

In an alternative embodiment, the system files and programming language necessary for performing the simulation may be loaded into the memory device of the computing device and performable by the processor, utilizing user inputs through the general user interface. In such an embodiment, there may be no need for communication through the network interface to communicate with external devices (e.g., servers) through a network router.

In the exemplary embodiment, the invention provides a novel system for a streamlined experience for all stakeholders, to healthcare professionals to industry representative to patients, making the system a valuable asset in the submissions, processing, management and renewal of urology supply prescriptions/orders.

The proposed design of the system includes three key components: an administrative/navigation panel 5, a back-end user module 6, and a front-end user module 8. These components work in unison to streamline user interaction, data management, and oversight capabilities within the system, enabling efficient workflow management and enhanced user experience.

In the exemplary embodiment, the administrative/navigation panel 5 provides oversight privileges through a login module 4, allowing system administrators to monitor and control both the back-end modules 6 and the front-end user modules 8. Administrators can access high-level information and manage critical functions such as user accounts, system settings, and overall system performance. The administrative/navigation panel 5 ensures smooth operation by providing real-time insights into system usage, urology supply prescriptions/orders, and front-end user engagement, making it a crucial part of the tool for maintaining operational efficiency and compliance. The panel 5 includes a review dashboard 18 that provides an overview of the prescriptions/orders submitted through the platform and any prescriptions/orders that are pending and need further action for submission through the platform. User authentication uses user name and password to secure access, with role-based permissions to restrict sensitive functions.

In the exemplary embodiment, the administrative/navigation panel 5 includes a setup new clinic module 10. The setup new clinic module 10 permits an administrator to create a new clinic by selecting the module 10. The setup new clinic module 10 will permit the user to access a create account module 12 allowing the user to provide the clinic name, clinic phone number, and the clinic address. The administrative/navigation panel 5 further includes an add users module 14 permitting front-end users access to the specific created clinic. The administrative/navigation panel 5 further includes a history module 30 to permit front-end users to review prescriptions/orders submitted through the platform, and a pending module 31 permitting the front-end users to view prescription/order currently pending within the system 1.

In the exemplary embodiment, the back-end user module 6 serves as a comprehensive management interface for users with administrative or operational roles. It includes a collection of at least four key tabs to streamline various aspects of the system, such as a dashboard module 18, an orders module 20, a clinics module 22, and a users module 24.

In the exemplary embodiment, the dashboard module 18 provides users with a high-level overview of ongoing activities. Users can view real-time statistics such as the total number of orders currently being processed, the total clinics engaged with the system, and the total number of users actively participating in the system. The dashboard module 18 serves as a central hub for key metrics, enabling quick assessment of system performance and engagement.

In the exemplary embodiment, the orders module 20 enables users to manage and track all orders processed through the system. The orders module 20 categorizes orders into submitted, pending, and fulfilled, providing users with full visibility over order status. The orders module 20 further identifies for each order the clinic name, the creator of the request, the patient, the date, and the status. This enables efficient order management, helping back-end users to monitor progress and ensure timely fulfillment. The orders module 20 integrates with vendor APIs to transmit prescription/orders directly, reducing manual handoffs. It includes error-handling protocols to alert users of discrepancies, such as missing HCPC codes, before final submission.

In the exemplary embodiment, the clinics module 22 is a tab that grants users the ability to view detailed information about each clinic currently engaged with the system. Users can access clinic-specific data, such as clinic participation rates, order history, and clinic-specific metrics, facilitating effective clinic management and engagement strategies. One skilled in the art would understand the applicant's design is not the exclusive embodiment.

In the exemplary embodiment, the users module 24 allows back-end users to view and manage front-end users who are actively engaging with the system. Back-end administrators can access user profiles, monitor user activity, and manage account settings or permissions as necessary. This ensures that user access is regulated and system integrity is maintained.

In the exemplary embodiment, the front-end user module 8 is designed to optimize the front-end user's experience by offering intuitive access to the system's core functionalities.

In the exemplary embodiment, the front-end user module 8 includes a login module 26; the login module 26 permits front-end user access to the various modules within the front-end user module.

In the exemplary embodiment, once the front-end user enters the system, the client page provides a history module 30, a pending module 31, an accounts module 32, a prescriber module 33, a settings module 34, and a new form order module 35.

In the exemplary embodiment, the history module 30 provides the name of the patient, the date of the order, the type of catheter or item of the order, the status associated with the order, and if the order was approved by the submitter. The history module 30 further provides the front-end user the ability to search prescriptions/orders and statistics of currently submitted orders or currently drafted/incomplete prescriptions/orders.

In the exemplary embodiment, the pending module 31 provides the user the ability to view any submitted orders that are currently pending.

In the exemplary embodiment, the accounts module 32 provides the user the ability to search and view their own account.

In the exemplary embodiment, the prescriber module 33 permits the user the ability to view the users tied to the account, invite a new user, denote a user as an administrator for the account, denote a user as a prescriber for the account and input their NPI number, and associated the user with one or several accounts.

In the exemplary embodiment, the settings module 34 permits the user the ability to view and update the clinic settings for the account.

In the exemplary embodiment, the new form order module 35 permits the user to be transported to an prescription/order form page module 36 to begin a new product request for a patient.

In the exemplary embodiment, the order form page module 36 includes various modules that enable front-end users to easily interact and manage their tasks, which generally includes a Documentation Module 40, a Patient Module 50, an Insurance Module 60, a Diagnosis Module 70, a Dispensing Module 90, a Product Module 100, a Supplies Module 120, a Validation Module 130, a Prescriber Module 140, a Review Module 150, and a Sign & Send Module 155.

In the exemplary embodiment, the new form module 35 enables the front-end user to create new prescription/order for urological supplies for a patient. The new form module 35 includes multiple sub-modules to facilitate detailed input, which will be described below.

In the exemplary embodiment, the documentation module 40 enables front-end users to upload medical records, including face sheets and chart notes related to the patient. In the exemplary embodiment, the documentation module 40 permits the user to upload a face sheet utilizing the face sheet module 42. In the exemplary embodiment, the documentation module 40 further permits the user to upload a chart notes module 44.

In the exemplary embodiment, the documentation module 40 is enhanced by a generative artificial intelligence and optical character recognition algorithm, which extracts relevant information from the uploaded documents notes and files. The algorithm pre-fills the fields across other modules, including a Patient Module 50, an Insurance Module 60, a Diagnosis Module 70, minimizing manual input and reducing errors, thereby significantly improving the efficiency of prescription/order creation. The AI model, based on a transformer architecture (e.g., BERT), is trained on a corpus of 10,000+ medical documents, including face sheets and chart notes, achieving an F1 score of 0.92 for entity extraction. It includes an error correction mechanism that suggests alternative inputs when extracted data deviates from expected formats (e.g., invalid diagnosis codes).

In the exemplary embodiment, the patient module 50 contains a plurality of selectable fields 52 for inputting patient information. One skilled in the art would understand the applicant's design is not the exclusive embodiment. If the plurality of selectable fields 52 are not automatically inputted through the documentation module, the front-end user will need to manually input the specific information.

In the exemplary embodiment, the insurance module 60 allows for the input of insurance details within the selectable fields 62. The insurance module further includes an insurance submission filing module 64 permitting the user to auto-fill the insurance information.

In the exemplary embodiment, the diagnosis module 70 provides a space to input patient diagnosis information. The diagnosis module 70 provides a primary diagnosis input module 72 allowing input of the diagnosis code associated with the episode of care for patient. The diagnosis module 72 further includes a secondary diagnosis code module 74 allowing input of the additional/ secondary diagnosis code associated with the episode of care for the patient if applicable.

In the exemplary embodiment, the diagnosis module 70 further includes a conditions input module 76 if applicable to the episode of care for the patient. In cases where the patient has history of urinary tract infections (“UTI”) and such information is important, the diagnosis module 70 further includes to support medical necessity for the product prescribed for the patient. In cases where indicating a latex allergy is important, the diagnosis module 70 further includes the ability to select whether the patient has or does not have history of UTI. The diagnosis module 70 further includes an additional notes module 82 with a typeable text area.

In the exemplary embodiment, the dispensing module 90 facilitates the input of information associated with when the product will be dispensed and for how long the patient will need the prescribed product. The dispensing module 90 includes a selectable start date module 92 indicating when a prescribed product will be dispensed.

In the exemplary embodiment, the dispensing module 90 further includes a selectable duration of need module 94 indicating how long the prescribed product will be needed.

In the exemplary embodiment, the product module 100 allows front-end users to select the appropriate urological supplies to be prescribed. The product module 100 includes a quick select module 102 and a marketplace module 108.

In the exemplary embodiment, the quick select module 102 allows the front-end user to choose a general product attributes if the front-end user does not desire select a specific product.

In the exemplary embodiment, the quick select module 102 includes a display. The display 104 provides a plurality of selectable icons 106 referring to general products such as “closed system,” “intermittent,” “male external,” and “foley.” Once an icon 105 is selected, frequently selected specification parameters will appear, allowing the front-end user to select required attributes about the selected product, include French size, length, firmness, and tip style.

In the exemplary embodiment, the quick select module 102 further includes a frequency module 107. The frequency module 107 permits the user to indicate an amount per day, an amount per month, and an amount per ninety days. One skilled in the art would understand the applicant's design is not the exclusive embodiment.

In the exemplary embodiment, the marketplace module 108 allows the front-end user to browse and select specialized parameters of the product to best fit the patient's needs.

In the exemplary embodiment, the selectable marketplace module 108 includes a display 110. The display 110 includes a plurality of selectable icons 112 referring to general products such as “closed system,” “intermittent,” “male external,” and “foley.” Once an icon 112 is selected, a detailed display of selectable parameter modules 114 will appear to provide the front-end user detailed customization. One skilled in the art would understand the applicant's design is not the exclusive embodiment.

In the exemplary embodiment, when the front-end user selects a product, the front-end user will further choose required attributes associated with the selected product and then utilize the add-to-cart module 116; the user will be taken to an accessories page module 118. The accessories page module 118 will display accessories associated with the selected product by the front-end user.

In the exemplary embodiment, the supplies module 120 leverages information input by the healthcare professional in generating the prescription/order and includes a customizable input module 122 providing a place to list any additional medical supplies required. One skilled in the art would understand the applicant's design is not the exclusive embodiment.

In the exemplary embodiment, the validation module 130 leverages information input by the healthcare professional in generating the prescription/order and compiles product information frequency, and diagnosis and then provides the user the ability to select qualifications from drop down lists, which are prepared and shown to the back-end user during an ordering process of the product. The validation module 130 includes a core method module. The core method module utilizes the main logic for the data shown on the validation screen, which lives in the Order.get_amendments() method.

In the exemplary embodiment, this method is responsible for returning a structured list of addendum/amendment entries—one per relevant product. Each amendment includes the diagnosis used for qualification, product group name, HCPC code (either selected or auto-derived), product name, usage frequency (per day/month/90 days), a formatted string of the usage values (e.g., “2 Per Day, 60 Per Month, 180 Per 90 Days”), and the qualification text, filled in with actual frequency numbers.

In the exemplary embodiment, the core method module further accesses the Order. get_amendments(). The core method module filters products; in this scenario, products from the “Foley” group are excluded entirely for the moment. The core method module then extracts product data. Specifically, for each product the core method module pulls, the core method module extracts the product group name, the Healthcare Common Procedure Coding System (“HCPC”) (or generates one using internal logic), and the product name. The core method module further searches for product options with the field “Frequency” and pulls the selected values, i.e., returning a list of three numbers: per day, per month, per 90 days. Once received, the core method module will generate a fallback code if needed; in this case, the core method module will call get_quick_hcpc_code (product_group, tip, diagnosis_code). The core method module will combine values into a readable string, such as: “2 Per Day, 60 Per Month, 180 Per 90 Days.” The core method module further retrieves a base qualification template from the system using the product's HCPC. The core method module will further substitute placeholders in the text like (day), (month), (ninety), or (90) with actual values from the product's frequency. Lastly, the core method module appends a dictionary for each product with all the fields described above. The validation module further includes a qualification logic module. The qualification logic module utilizes a table named AmendmentMappings to store the templates and structure needed for the qualification process. The qualification logic module will apply the process of accessing the class AmendmentMappings(models. Model): id=models. UUIDField(primary_key=True, default=uuid. uuid4, editable=False) created_at =models. DateTimeField(auto_now_add=True) modified_at=models. DateTimeField(auto_now=True)hcpc_code=models. CharField(max_length =10) qualification =models. TextField() options =models. JSONField(default=dict, blank=True).

The qualification logic module further accesses Fields: hcpc_code: The HCPCS code to which this mapping applies the qualification, essentially a string template used in the validation screen. It can include placeholders like (day), (month), (ninety), and (90), which are dynamically replaced. The qualification logic module further accesses a dictionary storing metadata or required configuration for this HCPC, used in the user interface. An example of how the qualification logic module flows is presented below.

### Example Qualification Flow

For a product with:
HCPC: A4351
Frequency: [2, 60, 180]
Qualification template: “Patient requires this product (day) times per day and (month) per month to manage their condition.”

The system will generate: “Patient requires this product 2 times per day and 60 per month to manage their condition.”

The validation module verifies compliance by cross-referencing inputs against a database of payer requirements, such as Medicare's Local Coverage Determinations (LCDs), ensuring addendums meet medical necessity criteria. The addendum is formatted as a JSON object with fields for HCPC code, frequency, diagnosis, and qualifications, exportable as a PDF for payer submission. CMS guidelines as set forth in the LCD. All commercial payers follow CMS, some less stringent than others.

In the exemplary embodiment, the validation module 130 includes a validation display page 132. The validation module 130 further includes an identifier module 134 positioned on the validation display page 132. The identifier module 134 provides the product name and frequency tab, the diagnosis tab, and the qualification tab. The identifier module 134 provides a drop-down menu within the qualification tab, permitting the user to select criteria that qualify the patient for the prescribed product.

In the exemplary embodiment, the prescriber module 140 collects prescriber information. The prescriber module 140 includes a plurality of inputting modules 142, such as an input prescriber name module, an input prescriber NPI number module, and a prescriber email. One skilled in the art would understand the applicant's design is not the exclusive embodiment.

In the exemplary embodiment, the review module 150 allows the front-end user to review the submitted responses through the review display module 152, which corresponds to the documentation module 40, the patient module 50, the insurance module 60, the diagnosis module 70, the dispensing module 90, the product module 100, the supplies module 120, the validation module 130, and the prescriber module 140.

In the exemplary embodiment, the sign and send module 155 allows the front-end user to finalize and submit the script. The sign and send module 155 permits the user to draw within a signature panel 156 the user's signature for authorization. One skilled in the art would understand the applicant's design is not the exclusive embodiment. In circumstances where the prescriber is not the user drafting the new order, the user will select the “Send to Prescriber for approval and signature” prior to submitting the order through an icon 157.

In the exemplary embodiment, the completed work order forms from the front-end user within the front-end user module 8 are sent to the back-end user through the back-end user module 6 for order processing.

In the exemplary embodiment, once the new order is submitted, the front-end user will be directed to an order received module 160. The order received module 160 provides the front-end user the ability to view a plurality of documents associated with the order, such as a documents uploaded to order module 162, an information & documents generated from order module 164, and a summary of all documents & information module 166.

In the exemplary embodiment, selecting the documents uploaded to order module 162 will direct the user to view those submitted for the new order.

In the exemplary embodiment, selecting the information & documents generated from order module 164 will direct the user to view the created written order.

In the exemplary embodiment, selecting the summary of all documents & information module 166 will direct the user to view the Medical Record Addendum/Amendment, which is previously generated by the validation module 130. The importance of this document, which is generated by the system, prevents the back and forth between of a prescriber and DMEPOS supplier if information is missing that is needed to comply with CMS guidelines and support medical necessity, which is required to fulfill the order and receive payment. This document generation will increase efficiency in office workflows with respect to generating and submitting prescriptions/orders and provide the patient with the necessary product at a quicker rate.

The order received module sends automated notifications to prescribers and the DMEPOS supplier upon submission, with a timestamped audit trail logging all actions for regulatory compliance.

It should be noted that various changes and modifications to the embodiments described herein will be apparent to those skilled in the art. Such changes and modifications may be made without departing from the spirit and scope of the present invention and without diminishing its intended advantages. For example, various embodiments of the systems and methods may be provided based on various combinations of the features and functions from the subject matter provided herein.

What is claimed is:

1. A prescription/order management system, comprises:
a processor and a server configured with a GPU to execute AI algorithms and optical character recognition, including natural language processing and machine learning models;
a computing device configured to run a web-based application accessible via a network;
a panel having a setup new clinic module enabling participant access by allowing administrators to onboard healthcare facilities with details like clinic name and address, the panel further having oversight privileges over a back-end user and a front-end user to monitor system performance and manage user accounts;
a back-end user module accessible by the back-end user having an orders module for receiving, tracking and fulfilling prescription/orders, including categorizing orders by status and displaying order details;
a front-end user module accessible by the front-end user having a new form module that enables the front-end user to create a new prescription/order form with assisted data entry that leverages generative artificial intelligence and optical character recognition, where a machine learning model extracts data from uploaded documents to pre-fill form fields; and
a validation module, the validation module compiling product information and qualifications by leveraging an inputted submission of the front-end user through the new form module to prepare a work order to present to the back-end user during an ordering process of the specific product and generating a medical record addendum with structured data, including HCPC codes, product selection, patient information, diagnosis, usage frequency and qualification, to ensure compliance.

2. The system of claim 1, wherein the panel further includes an add users module permitting the front-end user access to a specific created clinic by assigning user permissions and linking accounts to clinic profiles.

3. The system of claim 2, wherein the back-end user module further includes a dashboard module showing a total number of orders currently processed, along with metrics like order volume, clinic engagement, and user activity.

4. The system of claim 3, wherein the back-end user module further includes an orders module providing the back-end user an ability to manage and track all orders currently processed.

5. The system of claim 4, wherein the back-end user module further includes a clinics module providing the back-end user information about each clinic currently engaged with the system.

6. The system of claim 5, wherein the front-end user module further includes a patient module permitting the front-end user to submit patient information.

7. The system of claim 6, wherein the front-end user module further includes an insurance module permitting the front-end user to submit insurance information.

8. The system of claim 7, wherein the front-end user module further includes a diagnosis module allowing an input of a patient's condition.

9. The system of claim 8, wherein the front-end user module further includes a dispensing module.

10. The system of claim 9, wherein the dispensing module provides a frequency module indicating an amount per day, an amount per month, and an amount per ninety days of a product.

11. The system of claim 10, wherein the front-end user module further includes a product module.

12. The system of claim 11, wherein the product module includes a quick select module and a marketplace module.

13. The system of claim 12, wherein the quick select module permits limited selections and limited specification choices of the product.

14. The system of claim 13, wherein the marketplace module permits detailed customization of the product.

15. The system of claim 14, wherein the product module further includes a plurality of selectable icons referring to general medical products within both the quick select module and the marketplace module.

16. The system of claim 1, wherein the validation module further generates a medical record addendum/amendment through the provided information by the front-end user.

17. The system of claim 1, wherein the validation module includes a machine learning model based on a transformer architecture, trained on a dataset of historical prescription orders and medical documents to extract patient and prescription data from uploaded documents and pre-fill form fields, achieving an error reduction of up to 60% compared to manual entry.

18. The system of claim 1, wherein the validation module generates a medical record addendum by combining product data, frequency information, and diagnosis codes into a structured document that includes fields like HCPC code, usage frequency (e.g., 1 per day, 30 per month), and qualification criteria (e.g., “Patient requires this product due to neurogenic bladder”), reducing communication delays between prescribers and vendors by 40%.

19. The system of claim 1, wherein the front-end user module includes a product module with a quick select option for standard medical products with predefined specifications and a marketplace option for customized products, allowing users to specify parameters like size, tip type, and firmness, with AI-driven recommendations based on patient diagnosis.

20. The system of claim 1, wherein the system employs a cloud-based architecture with load balancing and redundant servers to support scalability, handling thousands of daily transactions across multiple clinics, and ensures data security through TLS 1.3 encryption and OAuth 2.0 authentication.

21. The system of claim 1, wherein the order received module generates automated notifications to prescribers and vendors upon order submission and maintains an audit trail of all actions for regulatory compliance.

22. The system of claim 1, wherein the validation module employs a rule-based algorithm to cross-reference inputs against payer-specific requirements, ensuring compliance with medical necessity criteria.
